(19) United States
(12) Patent Application Publication
(10) Pub. No.: US 2022/0071946 A1
(43) Pub. Date: Mar. 10, 2022

(54) Cannabis Treatment of Insomnia, Pain, and Skin Conditions

(71) Applicant: Alte Verde LLC, Huntington Beach, CA (US)

(72) Inventors: Shaun Land, Costa Mesa, CA (US); Wendy Hawkins, Costa Mesa, CA (US)

(21) Appl. No.: 17/191,470

(22) Filed: Mar. 3, 2021

Related U.S. Application Data
(60) Provisional application No. 62/984,691, filed on Mar. 3, 2020.

(51) Int. Cl. A61K 31/352; A61K 31/05; A61K 36/185; A61K 36/67; A61K 36/84; A61K 31/4045; A61K 36/48; A61K 36/77; A61K 36/47; A61K 36/81; A61K 36/36; A61K 31/675; A61P 25/20

(52) U.S. Cl. CPC A61K 31/352; A61K 31/05; A61K 36/185; A61K 36/67; A61K 36/84; A61K 31/4045; A61P 25/20; A61K 36/77; A61K 36/47; A61K 36/81; A61K 36/36; A61K 31/675; A61K 36/48

(57) ABSTRACT

A pharmaceutical composition for treating one or more symptoms of insomnia is disclosed herein. The pharmaceutical composition includes an amount of cannabidiol (CBD) and an amount of delta-9-tetrahydrocannabinol (THC), wherein the amount of CBD and the amount of THC are effective in treating insomnia. The pharmaceutical composition may include an amount of a selected herb and an amount of another cannabinoid. A pharmaceutical composition for treating one or more symptoms of chronic and acute pain using an amount of CBD and/or THC is also disclosed herein. The pharmaceutical composition may include an amount of another cannabinoid. A pharmaceutical composition for treating one or more symptoms of skin diseases and conditions using CBD and/or THC is also disclosed herein. The pharmaceutical composition may include an amount of another cannabinoid, an essential oil, a permeation enhancer, and/or an anti-bacterial/anti-viral agent.

### CROSS RELATED APPLICATIONS

This application claims priority to U.S. Provisional Patent Application No. 62/984,691 filed Mar. 3, 2020, which is incorporated by reference herein in its entirety.

### FIELD OF THE INVENTION

The present invention relates generally to pharmaceutical compositions for and methods of treating one or more symptoms of chronic or acute insomnia in a subject by administering the pharmaceutical composition to the subject wherein one or more symptoms of chronic or acute insomnia are treated in the subject. The pharmaceutical composition has an effective and controlled amount of cannabidiol (“CBD”) with and without tetrahydrocannabinol (“THC”), with and without other cannabinoids, and with and without select herbs.

The present disclosure further relates to pharmaceutical compositions for and methods of treating one or more symptoms of chronic or acute pain in a subject by administering the pharmaceutical composition to the subject wherein one or more symptoms of chronic or acute pain are treated in the subject. The pharmaceutical composition has an effective and controlled amount of CBD with and without THC, with and without other cannabinoids. In cases where subjects cannot tolerate THC or are not able to have THC in their system, the THC can be reduced to about 0 mg per dose.

The present disclosure further relates to pharmaceutical compositions for and methods of treating one or more symptoms of localized, chronic or acute pain and/or skin diseases/conditions in a subject by topically administering the pharmaceutical composition to the subject wherein one or more symptoms of localized, chronic or acute pain and/or skin diseases/conditions are treated in the subject. The pharmaceutical composition has an effective and controlled amount of CBD with and without THC, with and without other cannabinoids, and with and without essential oils.

### BACKGROUND OF THE INVENTION

Cannabinoids are a class of chemical compounds found in the Cannabis plant. The two primary cannabinoids contained in Cannabis are cannabidiol (“CBD”) and delta-9-tetrahydrocannabinol (“THC”). CBD lacks the equivalent psychoactive effects of THC. Studies have shown that CBD can be used to treat neurological disorders such as epilepsy, reduce inflammation and improve blood flow, reduce symptoms of arthritis, and cancer and eases symptoms of anxiety. THC has been shown to treat pain disorders, such as chronic and acute pain, as well as effectively fight viruses and cancer, and can cause sleepiness, drowsiness and other side effects beneficial to improving sleep and decreasing symptoms related to insomnia.

Outside the primary two cannabinoids contained in Cannabis, over 100 other cannabinoids have been identified in the Cannabis plant. A non-exhaustive list of these cannabinoids include cannabinol (“CBN”), cannabigerol (“CBG”), cannabichromene (“CBC”), cannabicyclol (“CBL”), cannabivarin (“CBV”), cannabidiolic acid (“CBDA”), and tetrahydrocannabinolic acid (“THCA”). These other cannabinoids have not been as extensively studied as the two primary cannabinoids, CBD and THC. As such, the effects of the other cannabinoids are not as well-known as CBD and THC. However, it is believed that these other cannabinoids also have numerous beneficial effects similar to the primary cannabinoids.

A. Chronic Insomnia Related Issues

According to the Nation Center for Health, One in Three US Citizens are suffering from chronic insomnia. Symptoms may include but are not limited to: trouble falling asleep; waking up throughout the night; trouble staying asleep or trouble returning to sleep; waking up too early; daytime sleepiness or grogginess; not feeling rested after a night's sleep; irritability; mood changes, such as feeling depressed; difficulty concentrating; problems with memory; and increase in mistakes and accidents.

The causes of insomnia can vary from emotional issues and stress, to long term medical conditions, including but not limited to: respiratory conditions, such as asthma, chronic obstructive pulmonary disease (COPD), and sleep apnea; congestive heart failure; diabetes; acid reflux; hyperthyroidism; fibromyalgia; pain; restless leg syndrome; menopause; urinary incontinence; stress, both physical and emotional; anxiety; depression; bipolar disorder; Alzheimer's disease; and Parkinson's disease.

For some people, certain medications and stimulants may cause chronic insomnia. These include but are not limited to: alcohol; antidepressants; beta-blockers; caffeine; chemotherapy drugs; cold and allergy medications containing pseudoephedrine; diuretics; illicit drugs, such as cocaine and other stimulants; nicotine; and stimulant laxatives. Certain lifestyle patterns may lead to chronic insomnia. These include but are not limited to: rotating shift work; frequent travel across multiple time zones, leading to jet lag; physical inactivity; frequent daytime napping; lack of routine for waking and sleeping; and poor sleeping environment.

Some of the prescription medications that are approved for treating insomnia include but are not limited to: zolpidem (Ambien); eszopiclone (Lunesta); zaleplon (Sonata); doxepin (Silenor); ramelteon (Rozerem); suvorexant (Belsomra); and temazepam (Restoril). Over the counter (OTC) sleep aid options may include: diphenhydramine (Benadryl); doxylamine succinate (Unisom SleepTabs); melatonin; valerian root; and chamomile tea.

The current generally available prescription medications have many negative side effects, including but not limited to: burning or tingling in the hands, arms, feet, or legs; changes in appetite; constipation; diarrhea; difficulty keeping balance; dizziness; daytime drowsiness; dry mouth or throat; gas; headache; heartburn; impairment the next day; mental slowing or problems with attention or memory; stomach pain or tenderness; uncontrollable shaking of a part of the body; unusual dreams; weakness; allergic reactions like anaphylaxis and angioedema; addiction; stroke; injury; and death.

Subjects do not have a safe and effective medication as an alternative. This disclosure covers pharmaceutical compositions and formulations that are not addictive, and much safer for subjects to take.

Also, subjects with chronic insomnia score much lower on the Anxiety, Depression, and Mood Scale (ADAMS), which is an instrument that is used by clinicians, doctors, and researchers to assess the level of anxiety, depression and mood in subjects with intellectual disabilities. ADAMS consists of questions grouped into five subscales, including (i) general anxiety, (ii) social avoidance, (iii) compulsive behavior, (iv) manic/hyperactive behavior, and (v) depressed mood. Each question is answered by a clinician/doctor on a four-point scale ranging from 0 (“not a problem”) to 3 (“severe problem”). In addition to subscale scores, the ADAMS yields a total score. Subjects with healthy sleep score much higher on ADAMS. Subjects on the pharmaceutical composition in this disclosure score higher on the ADAMS.

Select herbs, including but not limited to passion flower, kava, melatonin, valerian root and Erythrina Mulungu, have shown to improve sleep quality and decrease sleep issues related to insomnia.

B. Chronic and Acute Pain Related Issues

According to the National Center for Health Statistics (2006), approximately 76.2 million, one in every four Americans, have suffered from pain that lasts longer than 24 hours and millions more suffer from acute pain. Chronic pain is the most common cause of long-term disability.

The most common types of chronic and acute pain include: headache/migraine; postsurgical pain; post-trauma pain; back pain; cancer pain; arthritis pain; neurogenic pain (pain caused by nerve damage); and psychogenic pain (pain that isn't caused by disease, injury, or nerve damage). Many cases of chronic and acute pain are related to: back pain; arthritis, especially rheumatoid arthritis, and osteoarthritis; muscle tension/muscle injury; headache; multiple sclerosis; fibromyalgia; shingles; and nerve damage (neuropathy).

Some of the prescription medications that are approved for treating chronic and acute pain include: Codeine; Fentanyl (Actiq, Duragesic, Fentora, Abstral, Onsolis); Hydrocodone (Hysingla, Zohydro ER); Hydrocodone/acetaminophen (Lorcet, Lortab, Norco, Vicodin); Hydromorphone (Dilaudid, Exalgo); Meperidine (Demerol); Methadone (Dolophine, Methadose); Morphine (Kadian, MS Contin, Morphabond); Oxycodone (OxyContin, Oxaydo); Oxycodone and acetaminophen (Percocet, Roxicet); Oxycodone and naloxone; and Tramadol.

The current generally available prescription medications have many negative side effects and are addictive. Common negative side effects include: addiction/physical dependence; constipation; nausea; sedation, drowsiness, or clouded thinking; respiratory depression/slowed breathing; vomiting; tolerance; and death or injury from overdose.

Subjects do not have a safe and effective medication as an alternative. This disclosure covers pharmaceutical compositions and formulations that are not addictive, and much safer for subjects to take, with little to no negative side effects.

Also, subjects taking traditional prescription pain medications score much lower on the ADAMS. Subjects on the pharmaceutical composition in this disclosure will score much higher on the ADAMS.

C. Acute and Chronic Skin Disease/Condition Related Issues

According to the American Academy of Dermatology, 50 million Americans are affected by Acne annually. One in ten people will develop atopic dermatitis during their lifetime. 7.5 million people in the US have psoriasis. 16 million Americans have rosacea. Skin cancer is the most common cancer in the US with more than 9,500 people in the US diagnosed with skin cancer every day. These skin conditions result in pain, irritation, discomfort, and some forms of skin cancer are deadly. The American Academy of Dermatology has created a list of “skin conditions by the numbers” providing the above statistics and more, which is accessible on their webpage.

Subjects do not have alternative and highly effective pharmaceutical treatments for these skin conditions. This disclosure covers pharmaceutical compositions formulations that are not addictive, and much safer and effective for subjects to take, with little to no negative side effects.

### SUMMARY OF THE INVENTION

The present disclosure relates to a pharmaceutical composition having CBD and/or THC for treating one or more symptoms of chronic and acute insomnia in a subject. The present disclosure also relates to a method of treating one or more symptoms of chronic and acute insomnia in a subject by administering a pharmaceutical composition having CBD and/or THC to a subject. The pharmaceutical composition includes an effective amount of CBD and/or THC to treat one or more symptoms of chronic and acute insomnia in a subject. The pharmaceutical composition may further include an effective amount of other cannabinoids. The pharmaceutical composition may further include an effective amount of select herbs. The method includes administering the pharmaceutical composition to the subject wherein one or more symptoms of chronic or acute insomnia are treated in the subject, improving desirable datapoints related to duration and quality of sleep.

The present disclosure also relates to a pharmaceutical composition having CBD and/or THC for treating one or more symptoms of break-through pain, and chronic and acute pain in a subject. The present disclosure also relates to a method of treating one or more symptoms of break-through pain, and chronic and acute pain in a subject by administering a pharmaceutical composition having CBD and/or THC to a subject. The pharmaceutical composition includes an effective amount of CBD and/or THC to treat one or more symptoms of break-through pain, and chronic and acute pain in a subject. The pharmaceutical composition may further include an effective amount of other cannabinoids. The method includes administering the pharmaceutical composition to the subject wherein one or more symptoms of break-through, chronic or acute pain are treated in the subject, improving desirable datapoints related to pain intensity among others. The present disclosure also relates to replacing opioid-based drugs for subjects currently under pain management and/or addicted to opiates.

The present disclosure also relates to a pharmaceutical composition having CBD and/or THC for treating one or more symptoms of various skin diseases/conditions and localized chronic and acute pain in a subject. The present disclosure also relates to a method of treating one or more symptoms of various skin diseases/conditions and localized chronic and acute pain in a subject by administering a pharmaceutical composition having CBD and/or THC to a subject. The pharmaceutical composition includes an effective amount of CBD and/or THC to treat one or more symptoms of various skin diseases/conditions and localized chronic and acute pain in a subject. The pharmaceutical composition may further include an effective amount of other cannabinoids. The pharmaceutical composition may further include at least one selected essential oil. The pharmaceutical composition may further include a permeation enhancer and/or an anti-viral/anti-bacterial agent. The method includes topically administering the pharmaceutical composition to the subject wherein one or more symptoms of skin diseases/conditions and pain, either chronic or acute, are treated in the subject. The present disclosure also relates to replacing opioid-based drugs for subjects currently under pain management and/or addicted to opiates.

### DETAILED DESCRIPTION OF THE INVENTION

The invention now will be described more fully hereinafter, in which embodiments of the invention are described. This invention may, however, be embodied in many different forms and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” another element, it can be directly on the other element or intervening elements may be present there between. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section.

As used herein, the singular forms “a,” “an,” and “the,” are intended to include the plural forms as well, unless the context clearly indicates otherwise. It will be further understood that the terms “comprises” and/or “comprising,” “includes” and/or “including,” and “have” and/or “having,” when used in this specification, specify the presence of stated features, regions, integers, steps, operations, elements, and/or components, but do not preclude the presence or addition of one or more other features, regions, integers, steps, operations, elements, components, and/or groups thereof.

As used herein, the term “treat,” “treating,” “treated,” or “treatment” refers to mitigating, improving, relieving, or alleviating at least one symptom (such as a behavioral symptom) of a condition, disease or disorder in a subject, such as a human, or the improvement of an ascertainable measurement associated with a condition, disease or disorder.

As used herein, the term “clinical efficacy” refers to the ability to produce a desired effect in humans as shown through a Food and Drug Administration (FDA), or any foreign counterparts, clinical trial.

As used herein, the term “cannabinoids” refer to any and all cannabinoids present in Cannabis, including but not limited to THC, CBD, CBN, CBC, CBG, THCA, and CBDA. As used herein, the term “other cannabinoids” refers to all cannabinoids outside the primary two cannabinoids in Cannabis, THC and CBD, and includes all other cannabinoids present in Cannabis, including but not limited to CBN, CBC, CBG, THCA, and CBDA.

As used herein, the term “cannabidiol” or “CBD” refers to cannabidiol; cannabidiol prodrugs; pharmaceutically acceptable derivatives of cannabidiol, including pharmaceutically acceptable salts of cannabidiol, cannabidiol prodrugs, and cannabidiol derivatives. CBD includes, 2-[3-methyl-6-(1-methylethenyl)-2-cyclohexen-1-yl]-5-pentyl-1,3-benzenediol as well as to pharmaceutically acceptable salts, solvates, metabolites (e.g., cutaneous metabolites), and metabolic precursors thereof. The synthesis of CBD is described, for example, in Petilka et al., Helv. Chim. Acta, 52:1102 (1969) and in Mechoulam et al., J. Am. Chem. Soc., 87:3273 (1965), which are hereby incorporated by reference.

As used herein, the term “delta-9-tetrahydrocannabinol” or “THC” refers to THC; THC prodrugs; pharmaceutically acceptable derivatives of THC, including pharmaceutically acceptable salts of THC, THC prodrugs, and THC derivatives. The synthesis for THC is described, for example, in Trost, B. M., & Dogra, K., Synthesis of (−) -Delta9-trans-tetrahydrocannabinol: stereocontrol via Mo-catalyzed asymmetric allylic alkylation reaction, Organic letters, 9(5), 861-863 (2007), which is hereby incorporated by reference.

As used herein, the term “cannabinol” or “CBN” refers to the cannabinoid CBN; CBN prodrugs; pharmaceutically acceptable derivatives of CBN, including pharmaceutically acceptable salts of CBN, CBN prodrugs, and CBN derivatives.

As used herein, the term “cannabichromene” or “CBC” refers to the cannabinoid CBC; CBC prodrugs; pharmaceutically acceptable derivatives of CBC, including pharmaceutically acceptable salts of CBC, CBC prodrugs, and CBC derivatives.

As used herein, the term “cannabigerol” or “CBG” refers to the cannabinoid CBG; CBG prodrugs; pharmaceutically acceptable derivatives of CBG, including pharmaceutically acceptable salts of CBG, CBG prodrugs, and CBG derivatives.

As used herein, the term “orally administering,” “orally administered,” and “orally administer” refer to the subject consuming the pharmaceutical composition via a capsule, or sublingually as a composition via dropper or other measuring device.

As used herein, the term “topically administering,” “topically administered,” and “topically administer” refer to the subject applying the pharmaceutical composition to the skin.

Unless otherwise defined, all terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing.

The present disclosure generally relates to a pharmaceutical composition having CBD and/or THC for treating one or more symptoms of chronic or acute insomnia, localized, chronic, or acute pain, and/or localized, chronic, or acute skin diseases/conditions in a subject. The present disclosure also generally relates to a method of treating one or more symptoms of chronic or acute insomnia, localized, chronic, or acute pain, and/or localized, chronic, or acute skin diseases/conditions in a subject by administering a pharmaceutical composition having CBD and/or THC to a subject. In some embodiments, the pharmaceutical composition has an effective amount of CBD and/or THC to treat one or more symptoms of chronic or acute insomnia, localized, chronic, or acute pain, and/or localized, chronic, or acute skin diseases/conditions in a subject. In some embodiments, the pharmaceutical composition may further include select herbs. In some embodiments, the pharmaceutical composition may further include other cannabinoids. In some embodiments, the pharmaceutical composition may include essential oils. In some embodiments, the pharmaceutical composition may include a combination of other cannabinoids, herbs, and/or essential oils.

Clinical and preclinical data support the potential for CBD and THC in treating inflammation and pain, epilepsy, arthritis, cancer, neurological and sleep issues. Sublingual delivery of cannabinoids (e.g., CBD & THC) have benefits over capsule based oral dosing because it allows the drug to be absorbed through the sublingual glands directly into the bloodstream. This avoids first-pass liver metabolism, potentially enabling lower dosage levels of active pharmaceutical ingredients with a higher bioavailability and improved safety profile. Sublingual delivery also primarily avoids the gastrointestinal (GI) tract, lessening the opportunity for GI related adverse events and the potential degradation of CBD by gastric acid into THC, which can be associated with increased psychoactive effects. Moreover, sublingual delivery of cannabinoids increases the intensity and frequency of the desired somnolence events.

A. Pharmaceutical Composition for and Method of Treating Chronic Insomnia Related Issues

Chronic insomnia affects one in three people in the United States. This condition causes many physical and mental challenges for those affected by insomnia.

The present disclosure relates to a pharmaceutical composition having CBD and/or THC for treating one or more symptoms of chronic and acute insomnia in a subject. The present disclosure also relates to a method of treating one or more symptoms of chronic and acute insomnia in a subject by administering a pharmaceutical composition having CBD and/or THC to a subject. The pharmaceutical composition includes an effective amount of CBD and/or THC to treat one or more symptoms of chronic and acute insomnia in a subject. The pharmaceutical composition may further include an effective amount of other cannabinoids. The pharmaceutical composition may further include an effective amount of select herbs. The method includes administering the pharmaceutical composition to the subject wherein one or more symptoms of chronic or acute insomnia are treated in the subject, improving desirable datapoints related to duration and quality of sleep.

In some embodiments, the effective amount of CBD in the pharmaceutical composition can be between about 2 mg to about 25 mg per dose. The effective amount of THC in the pharmaceutical composition can be between about 0 mg per dose to about 17 mg per dose. In one embodiment, the effective amount of CBD is about 2 mg per dose, and the effective amount of THC is about 8 mg per dose. The pharmaceutical composition can be administered in a single daily dose or in some cases, two to three daily doses.

In some embodiments, selected herbs may be included in the pharmaceutical composition. The herbs may be selected from Passiflora incarnata (passion flower), Piper methysticum (kava), Valeriana officinalis (valerian), melatonin, Erythrina mulungu (Mulungu), Banisteriopsis caapi (Caapi), Ilex guayusa (Guayusa), Paullinia cupana (Guarana), C. angustifolia (Bobinsana), Jatropha macrantha (Huanarpo Macho), Withania somnifera (Ashwagandha), Silene undulata (Silene Capensis), and/or extracts from mushrooms (Psilocybin). This non-exhaustive list of selected herbs includes any other herbs shown to improve sleep quality, decrease sleep issues related to insomnia, and/or generally treat symptoms associated with insomnia.

In some embodiments, the herbs selected for the treatment of chronic and acute insomnia are kava and/or valerian. In these embodiments, the amount of valerian can be between 100 mg per dose and 300 mg per dose, and/or the amount of kava can be between 50 mg per dose and 150 mg per dose. In further embodiments, the amount of valerian is about 150 mg per dose, and/or the amount of kava is about 75 mg per dose.

In an alternative embodiment, the herbs selected for the treatment of chronic and acute insomnia are Mulungu and/or passion flower. In these embodiments, the amount of Mulungu can be between 2 mg per dose and 10 mg per dose, and/or the amount of passion flower can be between 1 mg per dose and 6 mg per dose. In other embodiments, the herbs selected for the treatment of chronic and acute insomnia are any combination of valerian, kava, passion flower, and Mulungu.

In some embodiments, an effective amount of other cannabinoids is included in the pharmaceutical composition. In some embodiments, the other cannabinoid is CBN. In these embodiments, the effective amount of CBN included in the pharmaceutical composition is between 2 mg per dose and 10 mg per dose.

In one specific embodiment, a formulation of a pharmaceutical composition for treating one or more symptoms of chronic and acute insomnia in a subject includes a composition of CBD, THC, valerian, and kava. In said embodiment, the formulation includes about 2 mg of CBD per dose, about 8 mg of THC per dose, about 150 mg of valerian per dose, and about 75 mg of kava per dose. In a further embodiment, the formulation also includes between 2 mg and 10 mg of CBN per dose.

The pharmaceutical composition can be formulated in a capsule, as an oil, and/or in liquid form. The pharmaceutical composition can be pharmaceutically produced to provide controlled drug delivery orally, with once- or twice- or three-times daily dosing. The pharmaceutical composition can be formulated in a solution for Intravenous (IV) administration. In some other embodiments, the pharmaceutical composition can be prepared for oral administration by preparing the pharmaceutical composition in a capsule or an oral solution. In some other embodiments, the IV preparation can be a diluted or undiluted solution. A person having ordinary skill in the art will appreciate how to formulate a capsule, oil, or liquid containing the pharmaceutical composition disclosed herein.

In some other embodiments, the pharmaceutical composition is formulated with a ratio of about 8 mg of CBD per ml up to about 16 mg of CBD per ml, with about 67 mg of THC per ml. In some embodiments, where the subject is hypersensitive to THC or cannot have THC in their system (e.g., for employment reasons), the THC may be reduced to about 0 mg of THC per ml. In some embodiments, MCT oil may be used as an inert base when the pharmaceutical composition is formulated as an oil. Other inert oils may be used in alternative embodiments.

Alleviating one or more symptoms of chronic or acute insomnia can include an improvement in a total score of ADAMS. In some embodiments, alleviating one or more symptoms of chronic or acute insomnia can include improvement in one or more subscales of ADAMS. In some embodiments, alleviating one or more behavioral symptoms of Autism Spectrum Disorder (ASD) can include improvement in one or more subscales of ADAMS.

In some embodiments, the one or more symptoms of chronic and acute insomnia include, but are not limited to the following symptoms: sleep quality; fatigue; problems with attention; concentration or memory (cognitive impairment); poor performance at school or work; moodiness or irritability; daytime sleepiness; impulsiveness or aggression; lack of energy or motivation; errors or accidents; concern or frustration about your sleep; quality of life; or any combination thereof. In some embodiments, a single symptom is alleviated. In some embodiments, any one of two, three, four, five, six, seven, eight, or more symptoms are alleviated.

In some embodiments, the CBD can be synthetic CBD. In some embodiments, the CBD can be purified CBD. In some embodiments, the CBD can be botanically derived. In some embodiments, the THC can be synthetic THC. In some embodiments, the THC can be purified THC. In some embodiments, the THC can be botanically derived. In some embodiments, the other cannabinoids can be botanically derived, purified, or synthetic.

The pharmaceutical composition can include diluents and carriers as well as other conventional excipients, such as wetting agents, preservatives, and suspending and dispersing agents. The pharmaceutical composition can also include a solubilizing agent, a permeation enhancer, a solubilizer, antioxidant, bulking agent, thickening agent, and/or a pH modifier.

A method of administering the pharmaceutical composition is also disclosed. The pharmaceutical composition can be orally administered via capsule, or in another embodiment, via sublingual administration via dropper under the subject's tongue. In embodiments where the pharmaceutical composition is formulated for an IV solution, the pharmaceutical composition is administered via IV drip or IV injection. The pharmaceutical composition may be administered in controlled doses once-, twice-, or three-times daily. In other embodiments, the pharmaceutical composition may be administered in as many doses as required to alleviate one or more symptoms of chronic or acute insomnia.

Data from the administration of embodiments of the pharmaceutical composition to adult subjects between the ages of 18 and 98 show the efficacy of the pharmaceutical composition in treating the symptoms of chronic and acute insomnia. Adult subjects include those with or without additional sleep aids. Most adult subjects were weaned off all other sleep aids, including prescription sleep aids. In all cases, the pharmaceutical composition effectively treated chronic and acute insomnia by 80% to 100% at an observational level and as reported by the adult subjects taking the pharmaceutical composition.

The data is based on comparing a baseline datapoint before administration of the pharmaceutical composition to the same datapoint after administration of the pharmaceutical composition. This comparison is noted as the change from baseline in each specific datapoint. The primary datapoint is the change from baseline in Average Nightly Total Sleep Time. Compared to the baseline Average Nightly Total Sleep Time, the pharmaceutical composition treated subjects should have a 50% to 100% reduction in symptoms of chronic or acute insomnia.

Secondary datapoints include the following: change from baseline in Unwanted Time Awake; change from baseline in Number of Awakenings During Sleep; change from baseline in Total Time Awake; change from baseline in Sleep Efficiency; change from baseline in Assessment of Sleep Quality; change from baseline in Participants' Impression of Daytime Functioning; change from baseline in the Insomnia Severity; change from baseline in Physical and Mental Component Scores; change from baseline in Health-related Quality of Life; Treatment Satisfaction; Clinical Global Impression of Improvement (CGI-I) in Insomnia; Patient Global Impression of Improvement (PGI-I) in Insomnia; change from baseline in Total Sleep Time; Number of Participants With Adverse Events (AEs) and Serious Adverse Events (SAEs) Based on Insomnia; Number of Participants With Treatment Emergent Adverse Events (TEAE); Number of Participants With Serious Adverse Events (SAEs); change from baseline in Blood Pressure (BP); change from baseline in Pulse Rate; change from baseline in Weight; and change from baseline in the ADAMS score.

Adult subjects have showed significant improvement in both the primary and many of the secondary datapoints from baseline. Adult subjects have shown a unanimous preference for this pharmaceutical composition versus previous prescribed and over-the-counter medications.

The pharmaceutical composition is well tolerated. One adult subject discontinued due to an increase in anxiety due to THC intolerance. No other adverse events led to discontinuation and no adverse events were considered severe. The most common adverse events were mild-moderate sleepiness after waking up. However, no subject experienced drug-related GI events during their treatment period. The data, in particular the improvements in primary and secondary datapoints, are very consistent, and immediate starting with the first dose.

Table 1 shows the results for numerous adult subjects who were administered the pharmaceutical composition for chronic and acute insomnia, called the “Insomnia Solution” or “solution” for the below table and accompanying examples.

TABLE 1
Results for Adult Subjects Administered the Pharmaceutical Composition for Chronic and Acute Insomnia.

| | | | | | | | CBD | THC | |
|---|---|---|---|---|---|---|---|---|---|
| | | | | | | | (mg) | (mg) | |
| | | | | | | | per | per | |
| Name | Age | Symptoms | Product | Note | Additives | Size | Size | Size | Efficacy |
| Pam W. | 60's | Cancer, | Insomnia | Insomnia - 1:6 | Herbs | 1 oz | 250 | 1000-2000 | 90-100% |
| | | Insomnia | Solution | with Valerian | | | | | insomnia |
| | | | | and Passion | | | | | improvement |
| | | | | Flower | | | | | |
| Caoilainn D. | 50's | Auto immune | Insomnia | NO | Herbs | 1 oz-4 oz | 250 | 1000 | 90-100% |
| | | issues, RA in | Solution | MELATONIN | | | | | insomnia |
| | | spine, | | | | | | | improvement |
| | | Fibromyalgia, | | | | | | | |
| | | major pain, | | | | | | | |
| | | extreme | | | | | | | |
| | | insomnia, | | | | | | | |
| | | allergic to | | | | | | | |
| | | melatonin, | | | | | | | |
| | | anemic, IBS | | | | | | | |
| Connie W. | 60's | Insomnia | Insomnia | | Herbs | 1 oz | 250-500 | 500-1000 | 100% insomnia |
| | | | Solution | | | | | | improvement |
| James L. | 70's | | | | | | | | 80%+ insomnia |
| | | | | | | | | | improvement |
| Keith L. | 60's | Severe back | Insomnia | | Herbs | 1 oz | 500-1000 | 500-1000 | 80%+ insomnia |
| | | pain, on pain | Solution | | | | | | improvement |
| | | prescriptions | | | | | | | |
| | | for 20+ years, | | | | | | | |
| | | had cancer, | | | | | | | |
| | | other issues | | | | | | | |
| Kevin D. | 60's | type 2 | Insomnia | | Herbs | 1 oz | 250-500 | 0-1000 | 90-100% |
| | | diabetes, | Solution | | | | | | insomnia |
| | | arthritis, | | | | | | | improvement |
| | | prostate | | | | | | | |
| | | cancer, colon | | | | | | | |
| | | cancer, did | | | | | | | |
| | | chemo | | | | | | | |
| Mike M. | 40's | Insomnia | Insomnia | | Herbs | 1 oz | 250 | 1000-2000 | 90-100% |
| | | | Solution | | | | | | insomnia |
| | | | | | | | | | improvement |
| Shaun L. | 40's | Insomnia | Insomnia | | Herbs | 1 oz | 250 | 250-2000 | 90%+ insomnia |
| | | | Solution | | | | | | improvement |
| Jeff W. | 40's | weight loss, | Insomnia | | Herbs | 1 oz | 500-1500 | 0 | 50-60% insomnia |
| | | insomnia, NO | Solution | | | | | | improvement |
| | | THC | | | | | | | |
| Wendy W. | 40's | hair falling | Insomnia | | Herbs | 1 oz | 250 | 1000 | 90-100% |
| | | out, insomnia | Solution | | | | | | insomnia |
| | | | | | | | | | improvement |
| Linda W. | 70's | Extreme | Insomnia | | Herbs | 1 oz | 250 | 1000 | 90-100% |
| | | depression, | Solution | | | | | | insomnia |
| | | anxiety, | | | | | | | improvement |
| | | insomnia | | | | | | | |
| Jim L. | 80's | Insomnia | Insomnia | CBN added | Herbs | 1 oz | 250 | 1000 | Significant |
| | | | Solution | (500 mg) | | | | | improvement |
| | | | | | | | | | over formula |
| | | | | | | | | | without CBN |
| Doug W. | 50's | Insomnia | Insomnia | CBN added | Herbs | 1 oz | 250 | 1000 | Positive |
| | | | Solution | (250 mg) | | | | | improvement |
| | | | | | | | | | over formula |
| | | | | | | | | | without CBN |
| Sylvia M. | 50's | Insomnia | Insomnia | CBN added | Herbs | 1 oz | 250 | 1000 | Mild |
| | | | Solution | (60 mg) | | | | | improvement |
| | | | | | | | | | over formula |
| | | | | | | | | | without CBN |
| Linda W. | 60's | Insomnia | Insomnia | CBN added | Herbs | 1 oz | 250 | 3000 | Positive |
| | | | Solution | (250 mg) | | | | | improvement |
| | | | | | | | | | over formula |
| | | | | | | | | | without CBN |
| Keith L. | 50's | Insomnia | Insomnia | CBN added | Herbs | 1 oz | 2000 | 2000 | Positive |
| | | | Solution | (350 mg) | | | | | improvement |
| | | | | | | | | | over formula |
| | | | | | | | | | without CBN |

Select examples of the Subject Monograph as reported by the subjects from Table 1 are provided below.

### Example 1: Subject Monograph as Reported by Subject Pam W.

This is the report regarding a subject in her 60's who has chronic insomnia due to multiple cancers. She has been taking the Insomnia Solution with select herbs for several months. She reported that, prior to taking the solution, she had great difficulty falling asleep. She also experienced waking up frequently, having problems going back to sleep after waking up, anxiety, brain racing, and overall poor sleep quality.

She reported that, starting with her first dose before bedtime, she experienced a drastic decline in her insomnia and symptoms, with an improvement of 90-100%. She was able to fall asleep faster, and seldom had sleep interruptions. She reported her overall sleep quality was high, and satisfaction in treatment was high. Subject is reported to be happier, more relaxed, and well rested, with much higher ADAMS and HRQL scores.

### Example 2: Subject Monograph as Reported by Subject Caoilainn D.

This is a report regarding a subject in her 50's who has chronic insomnia due to Auto Immune disease, Rheumatoid arthritis in her spine and Fibromyalgia with extreme insomnia, and IBS. She has been taking the CBD/THC solution for several months. She reported that within the first few nights of taking the solution, her insomnia was reduced by 90-100% and not have sleep interruptions. Her ADAMS and HRQL scores are much better with getting consistent, quality sleep.

### Example 3: Subject Monograph as Reported by Subject Connie W.

This is a report regarding a subject in her 60's who has chronic insomnia due to nerve pain, anxiety, IBS and couldn't leave the house due to anxiety. She has been taking the CBD/THC solution for several months. She reported that her insomnia was reduced by 100%, her nerve pain, IBS and anxiety was reduced by 90%. Her ADAMS and HRQL scores are much improved due to getting quality sleep.

### Example 4: Subject Monograph as Reported by Subject James L.

This is a report regarding a subject in his 70's who has chronic insomnia due to nerve pain in his legs and feet. He has been taking the CBD/THC solution for several months. He reported that taking the solution before bedtime immediately reduced his chronic insomnia by 80%+, and also helped reduce his nerve pain during sleep. His ADAMS and HRQL scores are much higher.

### Example 5: Subject Monograph as Reported by Subject Keith L.

This is a report regarding a subject in his 60's who has chronic insomnia due to chronic back pain. He was taking opiates and fentanyl patches for his pain for 20 years. On the CBD/THC solution, he was able to eliminate the opiates by 100% over 2 months and reported that taking the solution before bedtime immediately reduced his insomnia by 80%+. His ADAMS and HRQL scores are much higher due to getting consistent sleep and eliminating his long-term opiate addiction.

### Example 6: Subject Monograph as Reported by Subject Kevin D.

This is a report regarding a subject in his 60's who has chronic insomnia due to type 2 diabetes, arthritis, prostate cancer and colon cancer, and the effects of chemotherapy. He reported that taking the CBD/THC solution before bedtime reduced his insomnia by 90-100% and helped alleviate his chronic pain. His ADAMS and HRQL scores are much improved.

### Example 7: Subject Monograph as Reported by Subject Mike M.

This is a report regarding a subject in his 40's who has chronic extreme insomnia due to anxiety & stress. He was taking strong sleep medication prescriptions for years. He reported that by taking the CBD/THC solution before bedtime, he was able to eliminate his sleep medication prescriptions and reduced his insomnia by 90-100%. His ADAMS and HRQL scores are much improved.

### Example 8: Subject Monograph as Reported by Subject Shaun L.

This is a report regarding a subject in his 40's who has acute insomnia due to stress at work and travelling. He reported that by taking the CBD/THC solution before bedtime, ad-hoc when experiencing acute insomnia, he was able to eliminate prescription sleep medications and reduce his insomnia by 90%+. His ADAMS and HRQL score drastically improved compared to days following nights of insomnia.

### Example 9: Subject Monograph as Reported by Subject Jeff W.

This is a report regarding a subject in his 40's who has chronic insomnia due to stress. Due to his job requirements, he was taking the solution with just CBD and herbs, eliminating the THC in the solution. He reported that taking the solution before bedtime help reduce his insomnia by 50-60%. His ADAMS and HRQL scores are improved. However, he would prefer to take the CDB/THC formula to get even better results, if his job would allow it.

### Example 10: Subject Monograph as Reported by Subject Wendy W.

This is a report regarding a subject in her 40's who has chronic insomnia due to stress. She reported that by taking the CBD/THC solution before bedtime, she was able to reduce her insomnia by 90-100%. Her ADAMS and HRQL scores are much higher, and her anxiety and stress are also drastically reduced.

### Example 11: Subject Monograph as Reported by Subject Linda W.

This is a report regarding a subject in her 70's who has chronic insomnia due to depression, anxiety and stress. She reported that by taking the CBD/THC solution before bedtime, she was able to reduce her insomnia by 90-100%. Her ADAMS and HRQL scores are much higher, and her depression and anxiety overall are drastically reduced.

B. Pharmaceutical Composition for and Method of Treating Chronic and Acute Pain Related Issues

Chronic Pain affects one in four people in the United States. This condition causes many physical and mental challenges for those affected by chronic pain.

The present disclosure relates to a pharmaceutical composition having CBD and/or THC for treating one or more symptoms of break-through pain, and chronic and acute pain in a subject. The present disclosure also relates to a method of treating one or more symptoms of break-through pain, and chronic and acute pain in a subject by administering a pharmaceutical composition having CBD and/or THC to a subject. The pharmaceutical composition includes an effective amount of CBD and/or THC to treat one or more symptoms of break-through pain, and chronic and acute pain in a subject. The pharmaceutical composition may further include an effective amount of other cannabinoids. The method includes administering the pharmaceutical composition to the subject wherein one or more symptoms of break-through, chronic or acute pain are treated in the subject, improving desirable datapoints related to pain intensity among others. The present disclosure also relates to replacing opioid-based drugs for subjects currently under pain management and/or addicted to opiates.

In some embodiments, the effective amount of CBD can be between about 4 mg to about 16 mg per dose. The effective amount of THC can be between about 0 mg to about 8 mg per dose. The pharmaceutical composition can be administered in ad-hoc dosing (as needed for pain), or in scheduled dosing, consisting of one, two, three or four daily doses.

In some embodiments, an effective amount of other cannabinoids is included in the pharmaceutical composition. In some embodiments, the other cannabinoids are CBG and/or CBC. In these embodiments, the effective amount of CBG included in the pharmaceutical composition is between 2 mg per dose and 25 mg per dose. In these embodiments, the effective amount of CBC included in the pharmaceutical composition is between 2 mg per dose and 25 mg per dose. These embodiments may include either CBG or CBC or include both CBG and CBC. The addition of CBG and/or CBC provide additional anti-inflammatory and pain-relieving benefits.

The pharmaceutical composition can be formulated as a capsule, as an oil, or as a solution. The pharmaceutical composition can be pharmaceutically produced to provide controlled drug delivery orally, rectally, topically, or through IV. The pharmaceutical composition may be administered by ad-hoc dosing, once- or twice- or three-times or four-times daily dosing, or constant drip through IV.

In some embodiments, the pharmaceutical composition for oral administration can be prepared as a capsule, gel capsule or oral solution. In some embodiments, the pharmaceutical composition for rectal administration can be prepared as a capsule, or gel capsule. In some embodiments, the pharmaceutical composition for administration by IV drip can be prepared as a diluted or undiluted solution. In some embodiments, the pharmaceutical composition for administration by IV injection can be prepared as diluted or undiluted solution. In some other embodiments for treating one or more symptoms of localized break-through pain, and chronic and acute pain in a subject, the pharmaceutical composition may be topically administered to the subject wherein one or more symptoms of localized break-through, chronic or acute pain are treated in the subject. A person having ordinary skill in the art will appreciate how to prepare a capsule, gel capsule, oral solution, or oil containing the pharmaceutical composition disclosed herein.

In some embodiments, a solution of the pharmaceutical composition is formulated with a ratio of about 17 mg of CBD per ml up to about 67 mg of CBD per ml, with about 0 mg of THC per ml up to about 33 mg of THC per ml. In some embodiments, where the subject is hypersensitive to THC or cannot have THC in their system (e.g., for employment reasons), the THC may be reduced to about 0 mg of THC per ml.

Alleviating one or more symptoms of break-through, chronic or acute pain can include an improvement in a total score of ADAMS. In some embodiments, alleviating one or more symptoms of break-through, chronic or acute pain can include improvement in one or more subscales of ADAMS.

In some embodiments, the one or more symptoms is selected from the group consisting of general pain, specific pain, dull pain, sharp pain. These symptoms can result in mental and behavioral issues, such as general anxiety, social avoidance, compulsive behavior, manic/hyperactive behavior, irritability, lethargy, stereotypy, and inappropriate speech. The behavioral symptom that is alleviated can be any one of addiction behavior, general anxiety, social avoidance, compulsive behavior, manic/hyperactive behavior, mood, irritability, lethargy, stereotypy, emotional functioning, psychosocial health, socialization, reduced play and leisure, coping skills, internalizing behavior, externalizing behavior, quality of life, or any combination thereof. In some embodiments, a single symptom is alleviated. In some embodiment, two, three, four, five, six, seven, eight, or more symptoms are alleviated.

In some embodiments, the CBD can be synthetic CBD. In some embodiments, the CBD can be purified CBD. In some embodiments, the CBD can be botanically derived. In some embodiments, the THC can be synthetic THC. In some embodiments, the THC can be purified THC. In some embodiments, the other cannabinoids can be botanically derived, purified, or synthetic.

A method of administering the pharmaceutical composition is also disclosed. The pharmaceutical composition can be orally administered via capsule, or in another embodiment, via sublingual administration via dropper under the subject's tongue. In embodiments where the pharmaceutical composition is formulated for an IV solution, the pharmaceutical composition is administered via IV drip or IV injection. The pharmaceutical composition may be administered by ad hoc dosing, in controlled doses once-, twice-, or three-times daily, or by constant drip through IV. In other embodiments, the pharmaceutical composition may be administered in as many doses as required to alleviate one or more symptoms of chronic or acute pain.

Data from the administration of embodiments of the pharmaceutical composition to adult subjects between the ages of 18 and 98 shows the efficacy of the pharmaceutical composition in treating the symptoms of chronic and acute pain. The pain types treated include but are not limited to breakthrough pain (such as subjects taking opiate medication with opioid tolerance who experience persistent pain), general acute pain (such as visceral pain and non-visceral pain), and general chronic pain (such as neuropathic pain, chronic musculoskeletal pain, and any other type of general chronic pain).

Most adult subjects were weaned off other prescription and non-prescription pain medication, including all opiates. In all cases, the pharmaceutical composition effectively treated break-through, chronic and acute pain, at an observational level and as reported by the adult subjects taking the pharmaceutical composition.

The data is based on comparing a baseline datapoint before administration of the pharmaceutical composition to the same datapoint after administration of the pharmaceutical composition. This comparison is noted as the change from baseline in each specific datapoint. The primary datapoint is the change from baseline to at least 50% reduction in pain intensity. Compared to the baseline pain intensity score, the pharmaceutical composition treated subjects have a 75% to 100% reduction in symptoms of break-through, chronic or acute pain.

Secondary datapoints include the following: change from baseline in Physical Function; change from baseline in Emotional Function; change from baseline in Health-related Quality of Life (HRQL), including physical, psychological, and social datapoints; change from baseline in Participants' Impression of Daytime Functioning; Rescue Medication; Global Single-item assessment; Opioid Sparing; Sleep; Additional Measures; Treatment Satisfaction; Number of Participants With Serious Adverse Events (SAEs); Number of Participants With Treatment Emergent Adverse Events (TEAE); change from baseline in Blood Pressure (BP); change from baseline in Pulse Rate; change from baseline in Weight; and change from baseline in the ADAMS score.

Adult subjects have showed significant improvement in both the primary and many of the secondary datapoints from baseline. Adult subjects have shown a unanimous preference for the pharmaceutical composition versus previous prescribed medications.

The pharmaceutical composition is well tolerated. One adult subject discontinued due to an increase in anxiety due to THC intolerance. Some adult subjects did not like the psychoactive effect of the THC and reduced their THC dosage. No other adverse events led to discontinuation and no adverse events were considered severe. The most common adverse events were mild-moderate sleepiness or elation. However, no adult subject experienced drug-related GI events during the treatment period. The data, in particular the improvements in primary and secondary datapoints, are very consistent, and are often times immediate starting with the first dose.

Table 2 shows the results for numerous adult subjects who were administered the pharmaceutical composition for chronic and acute pain, called the “Pain Solution” or “solution” for the below table and accompanying examples.

TABLE 2
Results for Adult Subjects Administered the Pharmaceutical Composition for Chronic and Acute Pain.

| | | | | | | | | | |
|---|---|---|---|---|---|---|---|---|---|
| | | | | | | | CBD | THC | |
| Name | Age | Symptoms | Product | Note | Additives | Size | (mg) | (mg) | Efficacy |

| | | | | | | | | |
|---|---|---|---|---|---|---|---|---|
| Keith L. | 60's | Severe back pain, | Pain | | 1 oz | 2000-3000 | 125-500 | 100% |
| | | on pain | Solution | | | | | improvement |
| | | prescriptions for | | | | | | break-through |
| | | 20+ years, had | | | | | | pain; 90%+ |
| | | cancer, other | | | | | | improvement |
| | | issues | | | | | | chronic pain |
| Cristal C. | 42 | RA, Pain | Pain | | 1 oz | 250-500 | 500 | 90%+ |
| | | | Solution | | | | | improvement |
| | | | | | | | | chronic pain |
| Grace M. | 91 | extreme knee pain | Pain | | 1 oz | 250-500 | 250 | Pain manageable |
| | | | Solution | | | | | |
| Jill F. | 40's | Stage 4 Cancer | Pain | | 1 oz | 1500 | 750 | End of life chronic |
| | | | Solution | | | | | pain bearable |
| Addie S. | 94 | neuropathy, | Pain | | 1 oz | 250-1000 | 250-500 | 90%+ |
| | | anxiety, currently | Solution | | | | | improvement |
| | | taking 1:1 tincture | | | | | | chronic pain |
| | | 500/500. Need to | | | | | | |
| | | tweak formula a | | | | | | |
| | | bit for anxiety, | | | | | | |
| | | lost urinary | | | | | | |
| | | control. | | | | | | |
| Margaret W. | 65 | neck pain, | Pain | Was on | 1 oz-2 oz | 250-750 | 250-500 | 90%+ |
| | | arthritis. | Solution | Naproxen | | | | improvement |
| | | | | and | | | | chronic pain |
| | | | | Etodolac | | | | |
| Gilbert W. | 76 | Major back and | Pain | | 1 oz | 500 | 500 | 90%+ |
| | | spine pain | Solution | | | | | improvement |
| | | | | | | | | chronic pain |
| Stacie R. | 40's | thyroid cancer | Pain | | 1 oz | 1000-1500 | 250-500 | 90%+ |
| | | | Solution | | | | | improvement |
| | | | | | | | | chronic pain |
| Pam W. | 60's | Cancer, Insomnia | Pain | | 1 oz | 250-2000 | 250-2000 | 90%+ |
| | | | Solution | | | | | improvement |
| | | | | | | | | chronic pain |
| Angela C. | 30's | Chronic Pain - | Pain | | 1 oz | 250-750 | 500-750 | 90%+ |
| | | Fibromyalgia | Solution | | | | | improvement |
| | | | | | | | | chronic pain |
| Shaun L. | 40's | Back Pain | Pain | | 1 oz | 250 | 1250-1500 | 80%+ |
| | | | Solution | | | | | improvement |
| | | | | | | | | chronic pain |
| Sharon J. | 50's | Arthritis, back | Pain | 250 mg | 1 oz | 500 | 250 | Prefers formula |
| | | pain | Solution | CBC added | | | | with CBC (+10% |
| | | | | | | | | less pain) |
| James L. | 80's | Extreme back | Pain | 500 mg | 1 oz | 4000 | 1750 | Prefers formula |
| | | pain | Solution | CBG & 500 | | | | with CBG and |
| | | | | mg CBC | | | | CBC (+20% less |
| | | | | added | | | | pain) |
| Keith L. | 50's | Extreme back | Pain | 250 mg | 1 oz | 4000 | 1750 | Prefers formula |
| | | pain | Solution | CBG added | | | | with CBG (+10% |
| | | | | | | | | less pain) |
| Tony P. | 50's | Lower back, neck | Pain | 250 mg | 1 oz | 2000 | 750 | Prefers formula |
| | | pain. Fingers get | Solution | CBG & 250 | | | | with CBG and |
| | | numb | | mg CBC | | | | CBC (+15% less |
| | | | | added | | | | pain) |
| Josh S. | 40's | Broken arm, | Pain | 250 mg | 1 oz | 2000 | 3000 | Prefers formula |
| | | extreme pain | Solution | CBG & 250 | | | | with CBG and |
| | | | | mg CBC | | | | CBC (+10% less |
| | | | | added | | | | pain) |

Select examples of the Subject Monograph as reported by the subjects from Table 2 are provided below.

### Example 1: Subject Monograph of Break-Through and Chronic Musculoskeletal (Back) Pain, as Reported by Keith L.

This is the report regarding a subject in his 60's who has had chronic back pain due to spinal degeneration and injury who was on 8-10 opioids a day, as well as multiple Fentanyl patches per day for over 15 years. He suffered from opioid addiction, break-through pain, chronic pain, nausea, constipation and greatly reduced HRQL. He has been taking the CBD & THC oral solution for several months. As reported by Keith, he has now eliminated all opiates from his pain management regimen and is only taking the CBD & THC oral solution. Break-through pain was eliminated, and chronic pain was reduced by 90%+ to satisfactory levels for the subject. Constipation was eliminated. He reported that his overall pain levels are low, and his physical and emotion function was high, his HRQL is greatly improved and high satisfaction in treatment. Subject is very satisfied with his chronic pain management with the CBD/THC solution.

### Example 2: Subject Monograph of Chronic Arthritic Pain Due to Rheumatoid Arthritis, as Reported by Cristal C.

This is the report regarding a subject in her 40's who suffers from chronic pain throughout her body due to RA. She was undergoing chemotherapy (Remicade), prednisone, and refused taking opiates for pain management, and dealing with extreme pain and suffering from severe side effects of nausea, with very low HRQL. She was able to reduce her chronic pain by 90%+ with the CBD/THC oral solution and eliminate all other pain medications and chemotherapy. Her physician recently did her bloodwork, and her bloodwork came back perfect, where not only is the pain managed, but the RA showed as present and not active, with no flare-ups. She is now fully functioning physically and mentally. Her ADAMS and HRQL scores are much improved.

### Example 3: Subject Monograph of Chronic Knee Pain, as Reported by Grace M.

This is the report regarding a subject in her 90's suffering from chronic kneed pain, who couldn't even walk and would only take opioids when the pain was unbearable. Her physical and emotional functioning scores were very low. After taking the CBD/THC solution, her pain was manageable, and no longer needed to take opioids. Her ADAMS and HRQL scores were much improved.

### Example 4: Subject Monograph of Chronic Cancer-Related Pain, as Reported by Jill R.

This is a report regarding a subject in her 40's who suffered from stage 4 cancer. She was at end of life and taking narcotic pain medications, that were not helping with the break-through pain, and the chronic pain. Taking the CBD/THC solution, she was able to function mentally, and her chronic pain was bearable. Family reported that before she passed, the solution was instrumental in increasing her ADAMS and HRQL scores.

### Example 5: Subject Monograph of Chronic Neuropathy-Related Pain, as Reported by Addie S.

This is a report from a subject in her 40's who suffers from neuropathy and arthritis throughout the body, was barely walking, and also had loss of urinary control. On the CBD/THC solution, she was able to manage her chronic pain by 90%+, basically eliminating her pain. Her loss of urinary control was resolved as well. She now has a very favorable ADAMS and HRQL scores.

### Example 6: Subject Monograph of Chronic Arthritis-Related Pain, as Reported by Margaret W.

This is a report regarding a subject in her 60's who suffers from chronic arthritic pain in her neck. She had to stop taking NSAIDS (Naproxen and Etodolac) due to low kidney function. On the CBD/THC solution, she was able to manage her chronic pain by 90%+, eliminated kidney function issues, and greatly increased her ADAMS and HRQL scores.

### Example 7: Subject Monograph of Chronic Back and Spine-Related Pain, as Reported by Gilbert W.

This is a report for a subject in his 70's who suffers from chronic pain in the back and spine. On the CBD/THC solution, he was able to manage his chronic pain by 90%+, and greatly increase his ADAMS and HRQL scores.

### Example 8: Subject Monograph of Chronic Cancer-Related Pain, as Reported by Stacie R.

This is a report for a subject in her 40's who suffered from thyroid cancer and had chronic pain and nausea. On the CBD/THC solution, she was able to manage her chronic pain by 90%+, eliminate her nausea, and greatly increase her ADAMS and HRQL scores.

### Example 9: Subject Monograph of Chronic Cancer-Related Pain, as Reported by Pam W.

This is a report for a subject in her 60's who suffered from chronic pain due to breast and other cancers, as well as insomnia. On the CBD/THC solution, she was able to manage her chronic pain by 90%+, eliminated her nausea, eliminated her insomnia, and increased her ADAMS and HRQL scores.

### Example 10: Subject Monograph of Chronic Fibromyalgia-Related Pain, as Reported by Angela C.

This is a report for a subject in her 30's who suffered from chronic pain due to Fibromyalgia. She was taking multiple opiates daily and was addicted to opiates. She suffered many side-effects of the opiates, including fogginess, nausea and constipation. She also suffered from breakthrough pain. On the CBD/THC solution, she was able to manage her chronic pain by 90%+, use the solution for pain flare ups, and eliminate all opiates from her pain management regimen, and also eliminated the opiate related negative side effects. Her ADAMS and HRQL scores were much improved.

### Example 11: Subject Monograph of Acute and Chronic Musculoskeletal-Related Pain, as Reported by Shaun L.

This is a report for a subject in his 40's who suffered from acute and chronic pain due to a sports injury with a torn ACL and MCL. Immediately after the injury, and dosing 3-4 times per day, he took the CBD/THC solution for pain, and was able to reduce his pain by 80%+. Immediately after surgery to replace his ACL, he again took the CBD/THC solution 3-4 times daily instead of the prescribed opiates and was able to reduce his intense post-surgical pain by 80%+ and avoided taking any opiates. He was also able to work, and function levels were high. His ADAMS and HRQL scores were much improved.

C. Pharmaceutical Composition for and Method of Treating Chronic Pain and/or Skin Disease/Condition Related Issues

Chronic Pain affects one in four people in the United States. And skin diseases/conditions affect millions of Americans per year. These conditions cause many physical and mental challenges for those affected by chronic pain and/or skin diseases/conditions.

Clinical and preclinical data support the potential for CBD and THC in treating inflammation and pain, epilepsy, arthritis, cancer, neurological and sleep issues. Other data supports the potential for other cannabinoids, including CBG and CBC, in treating inflammation and pain, and as an anti-bacterial agent.

The present disclosure relates to a pharmaceutical composition having CBD and/or THC for treating one or more symptoms of various skin diseases/conditions and localized chronic and acute pain in a subject. The present disclosure also relates to a method of treating one or more symptoms of various skin diseases/conditions and localized chronic and acute pain in a subject by administering a pharmaceutical composition having CBD and/or THC to a subject. The pharmaceutical composition includes an effective amount of CBD and/or THC to treat one or more symptoms of various skin diseases/conditions and localized chronic and acute pain in a subject. The pharmaceutical composition may further include an effective amount of other cannabinoids. The pharmaceutical composition may further include at least one selected essential oil. The pharmaceutical composition may further include a permeation enhancer and/or an anti-viral and/or anti-bacterial agent. The method includes topically administering the pharmaceutical composition to the subject wherein one or more symptoms of skin diseases/conditions and pain, either chronic or acute, are treated in the subject. The present disclosure also relates to replacing opioid-based drugs for subjects currently under pain management and/or addicted to opiates.

The dosage amount applicable will depend upon the surface area of the skin and/or exposed tissue in which the pain and/or skin disease/condition exists. The dosage form (for example a gel, lotion, balm, or cream) may be selected for which it is suitable for the pain, disease, or condition and administration to the skin and/or exposed tissue. The dosage form may also be in such an amount and in such form that the pharmaceutical composition is in an effective dosage amount to treat said pain or disease/condition by penetration at the site of the skin and/or exposed tissue to be treated and is immediately available to transport (facilitate or cause the transport of) the pharmaceutical composition to the site of trauma and/or pathology to be treated, percutaneously into the skin (or exposed tissue) where the pharmaceutical composition resides and accumulates for a prolonged period, and which the pharmaceutical composition is in an effective non-toxic dosage amount to transport (facilitate or cause the transport of) upon administration, percutaneously into the skin or exposed tissue to the site of the pain, trauma and/or pathology.

In some embodiments, the pharmaceutical composition can be formulated as a liquid, balm, gel, lotion, or cream. In some embodiments, the pharmaceutical composition for topical use can be a liquid solution. In some embodiments, the pharmaceutical composition for topical use can be a balm. In some embodiments, the pharmaceutical composition for topical use can be a gel. In some embodiments, the pharmaceutical composition for topical use can be a cream. A person having ordinary skill in the art will appreciate how to formulate a liquid, balm, lotion, or cream containing the pharmaceutical composition disclosed herein.

In some embodiments, the effective amount of CBD in the pharmaceutical composition can be between about 250 mg to about 1500 mg per ounce of formulation (e.g., the lotion or balm). The effective amount of THC the pharmaceutical composition can be between about 250 mg to about 1500 mg per ounce. In other embodiments, the pharmaceutical composition is formulated with a ratio of about 250 mg of CBD per ounce up to about 750 mg of CBD per ounce, with about 250 mg of THC per ounce, up to about 750 mg of THC per ounce. The pharmaceutical composition can be topically administered in ad-hoc dosing, or in scheduled dosing, consisting of one, two, three or four daily doses.

In some embodiments, where the subject is hypersensitive to THC or cannot have THC in their system (e.g., for employment reasons), the THC may be reduced to about 0 mg of THC per ounce.

In some embodiments, an effective amount of other cannabinoids is included in the pharmaceutical composition. In some embodiments, the other cannabinoids are CBG and/or CBC. In these embodiments, the effective amount of CBG included in the pharmaceutical composition is between 60 mg per ounce and 500 mg per ounce of formulation. In these embodiments, the effective amount of CBC included in the pharmaceutical composition is between 75 mg per ounce and 250 mg per ounce. These embodiments may include either CBG or CBC or include both CBG and CBC. The addition of CBG and/or CBC has been shown to provide additional anti-inflammatory, pain-relieving, and anti-bacterial benefits. Additionally, these other cannabinoids, especially CBC, will increase the efficacy of the pharmaceutical composition in treating skin diseases/conditions.

In some embodiments, the pharmaceutical composition may further include Hypochlorous Acid (“HOCL”). HOCL is an anti-viral and anti-bacterial agent. HOCL adds a significant increase in the capability of the pharmaceutical composition to kill bacterial and viruses. In embodiments using HOCL, the HOCL will replace some or all of the water in lotion formulations of the pharmaceutical composition. In embodiments using HOCL, the pharmaceutical composition includes HOCL in an amount between 100 ppm and 200 ppm.

In some embodiments, the pharmaceutical composition may further include a permeation enhancer, such as dimethyl sulfoxide (DMSO). The permeation enhancer promotes the absorption of the pharmaceutical composition when topically administered by transiently enhancing the subject's skin permeability. In embodiments using a permeation enhancer like DMSO, 0.5% or less of the permeation enhancer is included in the pharmaceutical composition. For example, in specific embodiments using DMSO, the pharmaceutical composition includes less than 141 mg of DMSO per ounce.

In some embodiments, select essential oils may be included in the pharmaceutical composition. The essential oils to be selected from include, but are not limited to, sweet orange extract, lavender extract, bergamot, or menthol.

The pharmaceutical composition may further include a solubilizing agent, a permeation enhancer, a solubilizer, antioxidant, bulking agent, thickening agent, and/or a pH modifier.

Alleviating one or more symptoms of localized, break-through, chronic or acute pain can include an improvement in a total score of ADAMS. In some embodiments, alleviating one or more symptoms of localized break-through, chronic or acute pain can include improvement in one or more subscales of ADAMS.

Alleviating one or more symptoms of skin diseases/conditions can include an improvement in a total score of ADAMS. In some embodiments, alleviating one or more symptoms of localized break-through, chronic or acute pain can include improvement in one or more subscales of ADAMS.

In some embodiments, one or more symptoms is selected from the group consisting of general pain, specific pain, dull pain, sharp pain, and/or skin diseases/conditions. These symptoms can result in mental and behavioral issues, such as general anxiety, social avoidance, compulsive behavior, manic/hyperactive behavior, irritability, lethargy, stereotypy, and inappropriate speech. The behavioral symptom that is alleviated can be any one of addiction behavior, general anxiety, social avoidance, compulsive behavior, manic/hyperactive behavior, mood, irritability, lethargy, stereotypy, emotional functioning, psychosocial health, socialization, reduced play and leisure, coping skills, internalizing behavior, externalizing behavior, quality of life, or any combination thereof. In some embodiments, a single symptom is alleviated. In some embodiments, any one of two, three, four, five, six, seven, eight, or more symptoms are alleviated.

A method of administering the pharmaceutical composition is also disclosed. The pharmaceutical composition can be topically administered as a liquid, balm, gel, lotion, or cream. The pharmaceutical composition can be topically administered by ad hoc dosing or in controlled doses once-, twice-, or three-times daily. In other embodiments, the pharmaceutical composition may be topically administered in as many doses as required to alleviate one or more symptoms of localized chronic or acute pain or to treat the skin disease/condition.

Data from the administration of embodiments of the pharmaceutical composition to subjects between the ages of 17 and 98 shows the efficacy of the pharmaceutical composition in treating the symptoms of chronic and acute pain and acute and chronic skin diseases/conditions. The pain types treated include but are not limited to breakthrough pain (such as subjects taking opiate medication with opioid tolerance who experience persistent pain), general acute pain (such as visceral pain and non-visceral pain), and general chronic pain (such as neuropathic pain, chronic musculoskeletal pain, and any other type of general chronic pain). The skin diseases/conditions treated include, but are not limited to skin cancer, other cancers coming to the surface of the dermis, Psoriasis, Eczema, Atopic Dermatitis, injured dermis (i.e., surgical sites, accidents, burns, cuts, etc.), and Herpes.

Most subjects were weaned off of other prescription and non-prescription pain medication, including all opiates. In all cases, the pharmaceutical composition effectively treated localized, break-through, chronic and acute pain, at an observational level and as reported by the subjects taking the pharmaceutical composition.

Additionally, most subjects were weaned off of other prescription and non-prescription skin disease/condition treatments. In 75% of all cases, the pharmaceutical composition effectively treated chronic and acute skin diseases/conditions, at an observational level and as reported by the subjects taking the pharmaceutical composition.

The data is based on comparing a baseline datapoint before administration of the pharmaceutical composition to the same datapoint after administration of the pharmaceutical composition. This comparison is noted as the change from baseline in each specific datapoint. The primary datapoint for pain is the change from baseline to at least 50% reduction in pain intensity. The primary datapoint for skin conditions is the change from baseline to at least 50% reduction in the condition. Compared to the baseline pain intensity score, the pharmaceutical composition treated subjects have a 75% to 100% reduction in symptoms of localized, break-through, chronic or acute pain. Compared to the baseline condition score, the pharmaceutical composition treated subjects have a 75% to 100% reduction in symptoms of skin diseases/conditions. Some subjects were completely healed from chronic conditions.

Secondary datapoints include the following: change from baseline in Physical Function; change from baseline in Emotional Function; change from baseline in Health-related Quality of Life (HRQL), including physical, psychological, and social datapoints; change from baseline in Participants' Impression of Daytime Functioning; Rescue Medication; Global Single-item assessment; Opioid Sparing; Sleep; Additional Measures; Treatment Satisfaction; Number of Participants With Serious Adverse Events (SAEs); Number of Participants With Treatment Emergent Adverse Events (TEAE); change from baseline in Blood Pressure (BP); change from baseline in Pulse Rate; change from baseline in Weight; and change from baseline in the ADAMS score.

Subjects have showed significant improvement in both the primary and many of the secondary datapoints from baseline. Subjects have shown a unanimous preference for the pharmaceutical composition versus previous prescribed medications.

The pharmaceutical composition is well tolerated. One subject discontinued due to an increase in anxiety due to THC intolerance. No other adverse events led to discontinuation and no adverse events were considered severe. The most common adverse events were mild sleepiness or elation. However, no adult subject experienced drug-related GI events during the treatment period. The data, in particular the improvements in primary and secondary datapoints, are very consistent, and are often times immediate starting with the first dose.

Table 3 shows the results for numerous subjects who were administered the pharmaceutical composition for chronic and acute pain and/or to treat skin diseases/conditions, called the “Topical Solution” or “solution” for the below table and accompanying examples.

TABLE 3
Results for Adult Subjects Administered the Pharmaceutical Composition for Chronic and Acute Pain and/or Skin Diseases/Conditions.

| | | | | Product | | | | CBD | THC | |
|---|---|---|---|---|---|---|---|---|---|---|
| Name | Age | Symptoms | Product | Format | Note | Additives | Size | (mg) | (mg) | Efficacy |
| Keith L. | 60's | Severe back | Topical | Balm | Severe back | | 2 oz | 500 | 500 | 100% |
| | | pain, on pain | Solution | | pain. Was | | | | | improvement |
| | | prescriptions | | | using Fentanyl | | | | | break-through |
| | | for 20+ | | | patches and | | | | | pain; 90%+ |
| | | years, had | | | opioids to help | | | | | improvement |
| | | cancer, few | | | control pain. | | | | | chronic pain |
| | | other issues | | | Started using | | | | | |
| | | | | | pain balm 2-3 | | | | | |
| | | | | | times per day | | | | | |
| | | | | | and now does | | | | | |
| | | | | | not use any | | | | | |
| | | | | | prescriptions | | | | | |
| | | | | | or patches. | | | | | |
| | | | | | Pain balm | | | | | |
| | | | | | significantly | | | | | |
| | | | | | reduced the | | | | | |
| | | | | | pain and | | | | | |
| | | | | | swelling by at | | | | | |
| | | | | | least 90%. | | | | | |
| Allen L. | 40's | Chronic Pain | Topical | Lotion | Chronic pain | | 4 oz | 3000 | 250 | 70%+ |
| | | | Solution | | due to CRPS. | | | | | improvement |
| | | | | | Applies relax | | | | | chronic pain |
| | | | | | lotion to flare | | | | | |
| | | | | | up spots 3-4 | | | | | |
| | | | | | times per day | | | | | |
| | | | | | and relax | | | | | |
| | | | | | lotion took | | | | | |
| | | | | | away the | | | | | |
| | | | | | burning | | | | | |
| | | | | | sensation and | | | | | |
| | | | | | pain within 1- | | | | | |
| | | | | | 2 days. | | | | | |
| | | | | | Subject | | | | | |
| | | | | | applies | | | | | |
| | | | | | liberally every | | | | | |
| | | | | | day. | | | | | |
| Branden S. | 30's | professional | Topical | Balm | Extreme pain | IPO | 1 oz-4 oz | 2000-3000 | 2000-3000 | 90%+ |
| | | drummer | Solution | | in wrists and | | | | | improvement |
| | | with wrist | | | elbows due to | | | | | chronic pain |
| | | issues and | | | profession and | | | | | |
| | | elbow issues | | | car accident. | | | | | |
| | | | | | Started using | | | | | |
| | | | | | pain balm | | | | | |
| | | | | | daily and | | | | | |
| | | | | | within a few | | | | | |
| | | | | | days/weeks | | | | | |
| | | | | | saw 80% | | | | | |
| | | | | | difference in | | | | | |
| | | | | | mobility and | | | | | |
| | | | | | pain/stiffness | | | | | |
| | | | | | subsided. | | | | | |
| Calder M. | 40's | Extreme | Topical | Balm | Extreme pain | | 2 oz-4 oz | 250-1500 | 500-1500 | 80%+ |
| | | back pain | Solution | | in back from | | | | | improvement |
| | | | | | muscle strains. | | | | | chronic pain |
| | | | | | Started using | | | | | |
| | | | | | pain balm one | | | | | |
| | | | | | time per day | | | | | |
| | | | | | and saw an | | | | | |
| | | | | | immediate | | | | | |
| | | | | | difference. | | | | | |
| | | | | | Pain subsided | | | | | |
| | | | | | and mobility | | | | | |
| | | | | | returned. | | | | | |
| Alan D. | 40's | Pain in toe | Topical | Balm | Extreme pain | IPO | 4 oz | 1000 | 1000 | 100% |
| | | | Solution | | in toe. Started | | | | | improvement |
| | | | | | using pain | | | | | chronic pain |
| | | | | | balm on toe 2 | | | | | |
| | | | | | times per day | | | | | |
| | | | | | and within a | | | | | |
| | | | | | couple of | | | | | |
| | | | | | days, all pain | | | | | |
| | | | | | subsided. | | | | | |
| Ernie H. | 50's | Dislocated | Topical | Balm | Extreme pain | IPO | 2 oz-4 oz | 500-2000 | 500-2000 | 80%+ |
| | | shoulder, | Solution | | due to | | | | | improvement |
| | | Joint Pain, | | | dislocated | | | | | chronic pain |
| | | Extreme RA | | | shoulder. | | | | | |
| | | in hands | | | Rubs pain | | | | | |
| | | | | | balm on | | | | | |
| | | | | | shoulder 3 | | | | | |
| | | | | | times per day | | | | | |
| | | | | | to relieve pain | | | | | |
| | | | | | and reduce | | | | | |
| | | | | | swelling. Saw | | | | | |
| | | | | | immediate | | | | | |
| | | | | | results. | | | | | |
| | | | | | Shoulder is | | | | | |
| | | | | | completely | | | | | |
| | | | | | healed now. | | | | | |
| Grace | 91 | extreme | Topical | Balm | Pain in knee. | | 2 oz-4 oz | 250-500 | 250-500 | 90%+ |
| | | knee pain | Solution | | Very hard | | | | | improvement |
| | | | | | time walking, | | | | | chronic pain |
| | | | | | walks with a | | | | | |
| | | | | | cane. Started | | | | | |
| | | | | | using pain | | | | | |
| | | | | | balm on knee | | | | | |
| | | | | | 2 times per | | | | | |
| | | | | | day and within | | | | | |
| | | | | | a couple of | | | | | |
| | | | | | days was able | | | | | |
| | | | | | to put weight | | | | | |
| | | | | | on the leg and | | | | | |
| | | | | | walk without | | | | | |
| | | | | | the cane. Pain | | | | | |
| | | | | | has subsided | | | | | |
| | | | | | by at least | | | | | |
| | | | | | 90%. | | | | | |
| Melody P. | 40's | back and | Topical | Balm | Extreme back | | 2 oz-4 oz | 500-1000 | 500-1000 | 80%+ |
| | | disc issues | Solution | | and disc | | | | | improvement |
| | | | | | issues. Pain | | | | | chronic pain |
| | | | | | and | | | | | |
| | | | | | inflammation. | | | | | |
| | | | | | Starting using | | | | | |
| | | | | | pain balm 3 | | | | | |
| | | | | | times per day | | | | | |
| | | | | | and | | | | | |
| | | | | | inflammation | | | | | |
| | | | | | and pain | | | | | |
| | | | | | subsided by | | | | | |
| | | | | | 80% within 3 | | | | | |
| | | | | | days. | | | | | |
| Pam W. | 60's | Cancer, | Topical | Balm | | | 4 oz | 1000 | 1000 | 90%+ |
| | | Insomnia | Solution | | | | | | | improvement |
| | | | | | | | | | | chronic pain |
| Angela C. | 30's | Tom rotator | Topical | Balm | | | 2 oz | 500 | 500 | 90%+ |
| | | cuff in | Solution | | | | | | | improvement |
| | | extreme pain | | | | | | | | chronic pain; |
| | | | | | | | | | | Opiates |
| | | | | | | | | | | regiment |
| | | | | | | | | | | eliminated |
| Shaun L. | 40's | Back Pain | Pain | | | | 1 oz | 250 | 1250-1500 | 80%+ |
| | | | Solution | | | | | | | improvement |
| | | | | | | | | | | chronic pain |
| Keith S. | 40's | | Topical | Lotion | | | | | | 100% |
| | | | Solution | | | | | | | improvement |
| | | | | | | | | | | pre-cancerous |
| | | | | | | | | | | spots |
| Cindy B. | 50's | stage 3-4 | Topical | Lotion | Open blisters | IPO | 4 oz | 1000-2500 | 1000-2500 | 100% |
| | | breast cancer | Solution | | on breast due | | | | | improvement |
| | | on left side, | | | to breast | | | | | skin pain |
| | | red bumpy | | | cancer. | | | | | |
| | | blisters on | | | Extremely | | | | | |
| | | breast, | | | painful and | | | | | |
| | | extreme | | | swollen. Rubs | | | | | |
| | | pain, | | | relax lotion | | | | | |
| | | insomnia | | | around open | | | | | |
| | | | | | wound 3 times | | | | | |
| | | | | | per day. | | | | | |
| | | | | | Within 4-5 | | | | | |
| | | | | | days, blisters | | | | | |
| | | | | | started | | | | | |
| | | | | | diminishing | | | | | |
| | | | | | and going | | | | | |
| | | | | | away. Pain | | | | | |
| | | | | | also subsided. | | | | | |
| Darryl W. | 60's | Atopic | Topical | Lotion | Extremely | | 4 oz-16 oz | 1500-3000 | 250-1500 | 80%+ |
| | | Dermatitis/ | Solution | | painful/itchy/ | | | | | improvement |
| | | Eczema, Bad | | | burning Atopic | | | | | skin pain |
| | | skin | | | Dermatitis. | | | | | |
| | | condition, | | | Applies relax | | | | | |
| | | Extreme | | | lotion liberally | | | | | |
| | | itching | | | throughout the | | | | | |
| | | | | | day. Within 1- | | | | | |
| | | | | | 2 days noticed | | | | | |
| | | | | | significant | | | | | |
| | | | | | changes. | | | | | |
| | | | | | Burning and | | | | | |
| | | | | | itching | | | | | |
| | | | | | subsided by at | | | | | |
| | | | | | least 80%. | | | | | |
| Pang P. | 78 | Eczema | Topical | Lotion | Extreme | | 2 oz | 500 | 500 | 90%+ |
| | | | Solution | | eczema. | | | | | improvement |
| | | | | | Applies lotion | | | | | chronic pain |
| | | | | | on eczema | | | | | |
| | | | | | rash 1 time | | | | | |
| | | | | | per day and | | | | | |
| | | | | | noticed results | | | | | |
| | | | | | by the second | | | | | |
| | | | | | day. Redness, | | | | | |
| | | | | | dry skin and | | | | | |
| | | | | | itching | | | | | |
| | | | | | subsided | | | | | |
| | | | | | immediately. | | | | | |
| Pam W. | 60's | Cancer - | Topical | Lotion | Rash from | IPO | 1 oz-4 oz | 250-1000 | 500-5000 | 100% |
| | | Rashes | Solution | | cancer. | | | | | improvement |
| | | | | | Itching/painful. | | | | | skin pain |
| | | | | | Subject | | | | | |
| | | | | | applies relax | | | | | |
| | | | | | lotion 1-2 | | | | | |
| | | | | | times per day | | | | | |
| | | | | | and | | | | | |
| | | | | | pain/itching | | | | | |
| | | | | | subsides | | | | | |
| | | | | | immediately. | | | | | |
| | | | | | Subject now | | | | | |
| | | | | | uses relax | | | | | |
| | | | | | lotion only | | | | | |
| | | | | | when a new | | | | | |
| | | | | | rash appears. | | | | | |
| Stephanie T. | 50's | Benign | Topical | Lotion | Benign tumors | | 1 oz-4 oz | 1000-2250 | 500-1500 | 100% |
| | | salivary | Solution | | on face | | | | | improvement |
| | | tumors, has | | | affecting | | | | | nerve pain; |
| | | had 8 | | | nerves. | | | | | tumor growth |
| | | surgeries, | | | Applies relax | | | | | slowed |
| | | cannot have | | | lotion 3 times | | | | | |
| | | any more | | | per day on | | | | | |
| | | surgeries | | | tumors and | | | | | |
| | | | | | nerve | | | | | |
| | | | | | tingling/pain | | | | | |
| | | | | | has subsided. | | | | | |
| | | | | | Tumors are | | | | | |
| | | | | | not growing at | | | | | |
| | | | | | fast rate that | | | | | |
| | | | | | they were. | | | | | |
| | | | | | Subject started | | | | | |
| | | | | | noticing a | | | | | |
| | | | | | difference | | | | | |
| | | | | | within 1 week. | | | | | |
| Richie G. | 50's | extreme | Topical | Lotion | | 200 ppm | 4 oz | 1000 | 1000 | Reported |
| | | eczema | Solution | | | HOCL | | | | improved |
| | | | | | | | | | | results over |
| | | | | | | | | | | formula |
| | | | | | | | | | | without |
| | | | | | | | | | | HOCL (at |
| | | | | | | | | | | least 10%) |
| Emma H. | 17 | eczema | Topical | Lotion | | 200 ppm | 2 oz | 500 | 500 | Reported |
| | | | Solution | | | HOCL | | | | improved |
| | | | | | | | | | | results over |
| | | | | | | | | | | formula |
| | | | | | | | | | | without |
| | | | | | | | | | | HOCL (at |
| | | | | | | | | | | least 10%) |
| Jackie D. | 60's | plaque on | Topical | Lotion | 250 mg CBG | | 4 oz | 2000 | 2000 | Reported |
| | | fingernails | Solution | | added | | | | | improved |
| | | from arthritis | | | | | | | | results over |
| | | | | | | | | | | formula |
| | | | | | | | | | | without CBG |
| | | | | | | | | | | (at least 10%) |
| Doug W. | 50's | Extreme | Topical | Lotion | 500 mg CBG | 200 ppm | 4 oz | 1000 | 5000 | Reported |
| | | eczema, | Solution | | added | HOCL | | | | improved |
| | | psoriasis | | | | | | | | results over |
| | | | | | | | | | | formula |
| | | | | | | | | | | without CBG |
| | | | | | | | | | | and HOCL (at |
| | | | | | | | | | | least 10%) |
| Sharon J. | 50's | dry patches | Topical | Lotion | 250 mg CBG | | 2 oz | 500 | 500 | Reported |
| | | on body | Solution | | and 250 mg | | | | | improved |
| | | | | | CBC added | | | | | results over |
| | | | | | | | | | | formula |
| | | | | | | | | | | without CBG |
| | | | | | | | | | | and CBC (at |
| | | | | | | | | | | least 10%) |
| Pang P. | 70's | eczema | Topical | Lotion | 500 mg CBG | 200 ppm | 2 oz | 500 | 500 | Reported |
| | | | Solution | | and 250 mg | HOCL | | | | improved |
| | | | | | CBC added | | | | | results over |
| | | | | | | | | | | formula |
| | | | | | | | | | | without CBG, |
| | | | | | | | | | | CBC and |
| | | | | | | | | | | HOCL (at |
| | | | | | | | | | | least 10%) |
| Donna G. | 50's | eczema | Topical | Lotion | 500 mg CBG | 200 ppm | 4 oz | 1500 | 1500 | Reported |
| | | | Solution | | added | HOCL | | | | improved |
| | | | | | | | | | | results over |
| | | | | | | | | | | formula |
| | | | | | | | | | | without CBG |
| | | | | | | | | | | and HOCL (at |
| | | | | | | | | | | least 10%) |
| Nancy C. | 40's | red, itchy | Topical | Lotion | 250 mg CBG | 200 ppm | 2 oz | 500 | 500 | Reported |
| | | rash on | Solution | | added | HOCL | | | | improved |
| | | elbows and | | | | | | | | results over |
| | | fingers | | | | | | | | formula |
| | | | | | | | | | | without CBG |
| | | | | | | | | | | and HOCL (at |
| | | | | | | | | | | least 10%) |
| Chere C. | 40's | extreme dry, | Topical | Lotion | 250 mg CBG | | 2 oz | 500 | 500 | Reported |
| | | itchy patches | Solution | | and 250 mg | | | | | improved |
| | | on face | | | CBC added | | | | | results over |
| | | | | | | | | | | formula |
| | | | | | | | | | | without CBG |
| | | | | | | | | | | and CBC (at |
| | | | | | | | | | | least 10%) |
| Doug W. | 50's | Back pain | Topical | Balm | 250 mg CBG | | 4 oz | 1000 | 1000 | Reported |
| | | | Solution | | and 250 mg | | | | | improved |
| | | | | | CBC added | | | | | results over |
| | | | | | | | | | | formula |
| | | | | | | | | | | without CBG |
| | | | | | | | | | | and CBC (at |
| | | | | | | | | | | least 10%) |
| Pete R. | 50's | Knee surgery | Topical | Balm | 750 mg CBG | | 4 oz | 2000 | 2000 | Reported |
| | | | Solution | | added | | | | | improved |
| | | | | | | | | | | results over |
| | | | | | | | | | | formula |
| | | | | | | | | | | without CBG |
| | | | | | | | | | | (at least 10%) |
| Carol L. | 50's | Knee pain | Topical | Balm | 250 mg CBG | | 4 oz | 1000 | 1000 | Reported |
| | | | Solution | | added | | | | | improved |
| | | | | | | | | | | results over |
| | | | | | | | | | | formula |
| | | | | | | | | | | without CBG |
| | | | | | | | | | | (at least 10%) |
| Steve C. | 50's | Back and | Topical | Balm | 2000 mg CBG | | 4 oz | 2000 | 2000 | Reported |
| | | knee pain | Solution | | added | | | | | improved |
| | | | | | | | | | | results over |
| | | | | | | | | | | formula |
| | | | | | | | | | | without CBG |
| | | | | | | | | | | (at least 10%) |
| Michelle E. | 50's | Extreme | Topical | Balm | 2000 mg CBG | | 4 oz | 2000 | 2000 | Reported |
| | | lower back | Solution | | and 1000 mg | | | | | improved |
| | | pain | | | CBC added | | | | | results over |
| | | | | | | | | | | formula |
| | | | | | | | | | | without CBG |
| | | | | | | | | | | and CBC (at |
| | | | | | | | | | | least 10%) |

Select examples of the Subject Monograph as reported by the subjects from Table 3 are provided below.

### Example 1: Subject Monograph of Break-Through and Chronic Musculoskeletal (Back) Pain, as Reported by Keith L. (Pain)

This is the report regarding a subject in his 60's who has had chronic back pain due to spinal degeneration and injury who was on 8-10 opioids a day, as well as multiple Fentanyl patches per day for over 15 years. He suffered from opioid addiction, break-through pain, chronic pain, nausea, constipation and greatly reduced HRQL. He has been topically applying the CBD & THC Topical Solution for several months, once or more daily, as needed. As reported by Keith, he has now eliminated all opiates from his pain management regimen and is only taking the CBD & THC Pain Solution. Break-through pain was eliminated, and chronic pain was reduced by 90%+ to satisfactory levels for the subject. Constipation was eliminated. He reported that his overall pain levels are low, and his physical and emotion function was high, his ADAMS and HRQL scores also greatly improved and high satisfaction in treatment. Subject is very satisfied with his chronic pain management with the topical CBD/THC solution.

### Example 2: Subject Monograph of Chronic Nerve-Related Pain Due to CRPS, as Reported by Allen L. (Pain & Skin Issues)

This is the report regarding a subject in his 40's who suffers from extreme chronic pain throughout his body due to CRPS. He was undergoing chemotherapy, prednisone, and highly addicted to high levels of opiates for pain management and dealing with extreme pain and suffering from severe side effects of nausea, with very low HRQL. He was able to reduce his chronic back pain by 70%+ by topically applying the CBD/THC balm form of the solution. His skin flare-ups were also greatly reduced when applying the topical lotion form of the solution several times per day. His chronic symptoms have also been reduced overall. His ADAMS and HRQL scores also increased.

### Example 3: Subject Monograph of Chronic Arthritic Pain, as Reported by Branden S. (Pain)

This is the report regarding a subject in his 30's suffering from chronic elbow, wrist and hands pain due to arthritis. He refused to take opioids due to his religion and was dealing with constant pain and discomfort, which also affected his job where he had to use his hands constantly. The pain was immediately reduced by 90%+ when topically applying the CBD/THC balm form of the solution topically several times per day. And after several months of use, the arthritis was reduced. His ADAMS and HRQL scores increased drastically.

### Example 4: Subject Monograph of Chronic Cancer-Related Pain, as Reported by Calder M. (Pain)

This is a report regarding a subject in his 40's who suffered from chronic back pain from muscle strain due to his job. Topically applying the CBD/THC balm form of the solution once per day topically, his pain subsided by 80%+ and his mobility returned. His ADAMs and HRQL scores increased as well.

### Example 5: Subject Monograph of Chronic Pain, as Reported by Alan D. (Pain)

This is a report from a subject in his 50's who suffers from intermittent acute toe pain due to arthritis. He started topically applying the CBD/THC balm form of the solution twice per day, and within a couple of days, 100% of his pain subsided.

### Example 6: Subject Monograph of Acute Musculoskeletal Pain, as Reported by Ernie H. (Pain)

This is a report regarding a subject in his 50's who suffered from extreme pain in his shoulder due to dislocation. He topically applied the balm form of the formula to relieve pain and reduce swelling. He saw immediate results with 80%+ relief in pain, and his shoulder healed well.

### Example 7: Subject Monograph of Chronic Back and Spine-Related Pain, as Reported by Grace M. (Pain)

This is a report for a subject in his 90's who suffers from chronic pain in her knee. She had a very hard time walking and used a cane. Topically applying the CBD/THC balm form of the solution 2 times per day she was able to put weight on the leg and walk without the cane within 2 days. Pain has subsided by at least 90%. Her ADAMS and HRQL scores greatly increased.

### Example 8: Subject Monograph of Chronic Back/Disc Pain, as Reported by Melody P. (Pain)

This is a report for a subject in her 40's who suffered from back and disc related chronic pain. Topically applying the CBD/THC balm form of the solution, she was able to manage her chronic pain by 80%+, within three days. Her ADAMS and HRQL scores were also improved.

### Example 9: Subject Monograph of Chronic Cancer-Related Pain, as Reported by Pam W. (Pain)

This is a report for a subject in her 60's who suffered from chronic pain due to breast and other cancers, as well as insomnia. On the CBD/THC solution, she was able to manage her chronic pain by 90%+, eliminated her nausea, eliminated her insomnia, and increased her ADAM and HRQL scores.

### Example 10: Subject Monograph of Chronic Fibromyalgia-Related Pain, as Reported by Angela C. (Pain)

This is a report for a subject in her 30's who suffered from chronic pain due to Fibromyalgia. She was taking multiple opiates daily and was addicted to opiates. She suffered many side-effects of the opiates, including fogginess, nausea and constipation. She also suffered from breakthrough pain. Topically applying the CBD/THC balm form of the solution, she was able to manage her chronic pain by 90%+, use the solution for pain flare ups, and eliminate all opiates from her pain management regimen, and also eliminated the opiate related negative side effects. Her ADAMS and HRQL scores were also much improved.

### Example 11: Subject Monograph of Acute and Chronic Musculoskeletal-Related Pain, as Reported by Shaun L. (Pain)

This is a report for a subject in his 40's who suffered from acute and chronic pain due to a sports injury with a torn ACL and MCL. Immediately after the injury, he applied the CBD/THC balm form of the solution 3-4 times per day and was able to reduce his pain by 80%+. Immediately after surgery to replace his ACL, he again topically applied the CBD/THC solution 3-4 times daily instead of the prescribed opiates and was able to reduce his intense post-surgical pain by 80%+ and avoided taking any opiates. His knee also healed 20% faster than expected, and his surgical sites also healed faster than expected. His ADAMS and HRQL scores were also much improved.

### Example 12: Subject Monograph of Pre-Cancerous Skin Condition, as Reported by Keith S. (Cancer/Skin)

This is a report for a subject in his 40's who was diagnosed with dark, pre-cancerous spots on his face and nose. He topically applied the CBD/THC lotion form of the solution 2 times per day over a 2-week period, and all pre-cancerous spots diminished by at least 75% in color and size. Further treatment resulted in the spots diminishing by 100%.

### Example 13: Subject Monograph of Breast-Cancer Related Skin Condition, as Reported by Cindy B. (Cancer/Skin)

This is a report for a subject in her 50's who had stage ¾ breast cancer, with persistent and worsening, open, red, bumpy blisters on her affected breast and was in extreme pain. By topically applying the CBD/THC lotion form of the solution on the affected area 3 timers per day, her blisters diminished and went away in 5 days, and the pain also subsided. She continues to topically apply the lotion form of the solution to her breast as her tumors are also shrinking. Her ADAMS and HRQL scores greatly increased.

### Example 14: Subject Monograph of Atopic Dermatitis & Eczema Related Skin Condition, as Reported by Darryl W. (Skin)

This is a report for a subject in his 60's who suffered from extreme and painful atopic dermatitis and eczema for years, with no resolution. His skin was extremely painful, itch, and burning 24×7. His ADAMS and HRQL scores were very low. After topically applying the CBD/THC lotion form of the solution liberally to the affected areas of his limbs and torso, several times per day, the burning and itching subsided by at least 80%. And his ADAMS and HRQL scores drastically increased.

### Example 15: Subject Monograph of Extreme-Eczema Related Skin Condition, as Reported by Pang P. (Skin)

This is a report for a subject in his 70's with extreme eczema. By topically applying the CBD/THC lotion form of the solution once per day, he noticed immediate results and by the second day, he redness, dry skin and itching subsided by 90%+.

### Example 16: Subject Monograph of Cancer Related Skin Issues, as Reported by Pam W. (Skin)

This is a report for a subject in her 60's who suffered from painful and itchy rashes from cancer. By topically applying the CBD/THC lotion form of the solution twice per day, the pain and itching subsided immediately and eliminated the rash. She now applies the lotion form of the solution only if a new rash appears. Her ADAMS and HRQL scores were greatly improved.

### Example 17: Subject Monograph of Benign Tumors Related Skin Issues, as Reported by Stephanie T. (Skin)

This is a report for a subject in her 50's who suffered from fast growing benign tumors on her face, which were also affecting her nerves. By topically applying the CBD/THC lotion form of the solution, the nerve tingling & pain subsided. The tumor growth was also abruptly slowed within 1 week.

I claim:

1. A pharmaceutical composition for treating one or more symptoms of insomnia comprising:
an amount of cannabidiol; and
an amount of delta-9-tetrahydrocannabinol;
wherein said amount of cannabidiol and said amount of delta-9-tetrahydrocannabinol are effective in treating insomnia.

2. The pharmaceutical composition for treating one or more symptoms of insomnia of claim 1, further comprising:
at least one herb.

3. The pharmaceutical composition for treating one or more symptoms of insomnia of claim 2, wherein said at least one herb is selected from passion flower, kava, valerian, melatonin, Mulungu, Caapi, Guayusa, Guarana, Bobinsana, Huanarpo Macho, Ashwagandha, Silene Capensis, and/or Psilocybin.

4. The pharmaceutical composition of claim 2, wherein said at least one herb are valerian and kava.

5. The pharmaceutical composition for treating one or more symptoms of insomnia of claim 1, further comprising:
at least one other cannabinoid.

6. The pharmaceutical composition for treating one or more symptoms of insomnia of claim 5, wherein said at least one other cannabinoid is CBN.

7. The pharmaceutical composition of claim 1, wherein said amount of cannabidiol is between 2 mg per dose and 25 mg per dose, and wherein said amount of delta-9-tetrahydrocannabinol is between about 0 mg per dose and 17 mg per dose.

8. The pharmaceutical composition of claim 7, further comprising kava and valerian.

9. The pharmaceutical composition of claim 8, wherein said kava is present in an amount between 50 mg per dose and 150 mg per dose, and wherein said valerian is present in an amount between 100 mg per dose and 300 mg per dose.

10. The pharmaceutical composition of claim 1, wherein said amount of cannabidiol is 2 mg per dose and said amount of delta-9-tetrahyrdocannabinol is 8 mg per dose.

11. The pharmaceutical composition of claim 10, further comprising an amount of kava and an amount of valerian, wherein said amount of kava is 75 mg per dose and said amount of valerian is 150 mg per dose.

12. A pharmaceutical composition for treating one or more symptoms of insomnia comprising:
an amount of cannabidiol;
an amount of delta-9-tetrahydrocannabinol;
an amount of kava; and
an amount of valerian;
wherein said amount of cannabidiol, said amount of delta-9-tetrahydrocannabinol, said amount of kava, and said amount of valerian are effective in treating insomnia.

13. The pharmaceutical composition for treating one or more symptoms of insomnia of claim 12, wherein said amount of cannabidiol is between 2 mg per dose and 25 mg per dose, said amount of delta-9-tetrahydrocannabinol is between 0 mg per dose and 17 mg per dose, said amount of kava is between 50 mg per dose and 150 mg per dose, and said amount of valerian is between 100 mg per dose and 300 mg per dose.

14. The pharmaceutical composition for treating one or more symptoms of insomnia of claim 12, wherein said amount of cannabidiol is 2 mg per dose, said amount of delta-9-tetrahydrocannabinol is 8 mg per dose, said amount of kava is 75 mg per dose, and said amount of valerian is 150 mg per dose.

15. The pharmaceutical composition for treating one or more symptoms of insomnia of claim 13, further comprising an amount of CBN, wherein said amount of CBN is between 2 mg per dose and 10 mg per dose.

16. A method of treating insomnia in a subject, the method comprising:
administering an effective amount of a pharmaceutical composition to said subject experiencing symptoms of insomnia, wherein said pharmaceutical composition comprises: an amount of cannabidiol and an amount of delta-9-tetrahydrocannabinol.

17. The method of treating insomnia in a subject of claim 16, wherein said pharmaceutical composition further comprises an amount of kava and an amount of valerian.

18. The method of treating insomnia in a subject of claim 17, wherein said amount of cannabidiol is between 2 mg and 25 mg per dose, said amount of delta-9-tetrahydrocannabinol is between 0 mg and 17 mg per dose, said amount of kava is between 50 mg and 150 mg per dose, and said amount of valerian is between 100 mg and 300 mg per dose.

19. The method of treating insomnia in a subject of claim 17, wherein said amount of cannabidiol is 2 mg per dose, said amount of delta-9-tetrahydrocannabinol is 8 mg per dose, said amount of kava is 75 mg per dose, and said amount of valerian is 150 mg per dose.

20. The method of treating insomnia in a subject of claim 16, wherein said pharmaceutical composition is administered orally.
